(19) United States
(12) Patent Application Publication
(10) Pub. No.: US 2024/0041654 A1
(43) Pub. Date: Feb. 8, 2024

(54) ASSEMBLY FOR LASER TREATMENT OF OCULAR OPACITIES

(71) Applicant: Carl Zeiss Meditec AG, Jena (DE)

(72) Inventors: Martin Hacker, Jena (DE); Alexander Gratzke, Jena (DE); Stephan Merkel, Jena (DE)

(21) Appl. No.: 18/246,603

(22) PCT Filed: Sep. 24, 2021
(86) PCT No.: PCT/EP2021/076319

§ 371 Date: Mar. 24, 2023

(30) Foreign Application Priority Data
Sep. 25, 2020 (DE) 10 2020 212 084.6

(51) Int. Cl. A61F 9/008

(52) U.S. Cl. CPC A61F 9/00825; A61F 2009/00874; A61F 2009/00846; A61F 2009/00851

(57) ABSTRACT

The assembly for laser treatment of ocular opacities consists of: a measurement system for obtaining depth information regarding ocular structures; a laser system; an eye-tracker unit; a display unit; and a control-and-operating unit. According to the invention, the control-and-operating unit is designed to determine, from the depth profiles, the depth of ocular structures relative to the depth of the laser focus, and, in particular for the retina and the capsular bag, to determine a blocked zone for the laser treatment. Furthermore, the control-and-operating unit is designed to generate, at least for the blocked zones of the retina and capsular bag and the laser focus, at least one tag in each case, the characteristic of which corresponds to the particular depth in the eye, in order to display these tags on the display unit and to overlay them with the live image. The invention relates to a partially automated therapy apparatus for laser treatment of ocular opacities in which two-dimensional views of the eye are combined with three-dimensional imaging from the measurement system.

### TECHNICAL FIELD

The present invention relates to an arrangement for laser treatment of eye opacification.

### BACKGROUND

The vitreous humor consists of a usually clear, gel-like substance in the interior of the eye between the lens and the retina. In youth, the vitreous humor is completely clear and has contact with the retina. Over the course of a lifetime, the vitreous humor liquefies and increasingly detaches from the retina; this is referred to as posterior vitreous detachment. This is a normal aging process which usually occurs after the age of 50. The detached vitreous humor components come together in the interior of the eye and the framework substances and concentrations of the vitreous humor are rendered visible to the patients. Since they may also move across the visual field, they are also referred to as floaters. Often, as a cause of floaters, membrane-like structures are also present on the posterior side of the vitreous humor following the vitreous humor detachment, sometimes even the remains of blood should retinal injury have occurred during the vitreous humor detachment. In rare cases, floaters may also be present as crystal-like precipitates in the vitreous humor in the case of metabolic problems.

Even if floaters usually do not have a pathological cause, they are not as harmless as generally assumed because they can impair, sometimes significantly impair, the quality of life and also work productivity of the affected parties.

This opacification is perceived especially against a bright background, for example when working on a computer, when reading or when looking at the blue sky or snow, and disturb the visual faculty. Floaters that are flung into and out of the central field of vision as a result of the reading movements when reading can be particularly bothersome.

Since these often have the form of a “flying gnat”, they are described using the technical term “mouches volantes”—which comes from the French. However, the opacification may also have different shapes, for example be branch-, ring- or star-shaped, or else be present as point clouds. In the following text, the term “floater” is used for the vitreous humor opacification to be treated, irrespective of its type or form.

In general, floaters do not disappear without treatment because the immune system does not recognize these as abnormal and therefore does not destroy these. However the affected parties can hardly ignore or overlook them. Certain floater types, such as those caused by residual blood following retinal bleeding, are partly resorbed by the body again, even if this often takes weeks or months.

In what is known as vitrectomy, the vitreous humor is partly (core vitrectomy) or completely comminuted, aspirated and removed after the eye has been opened up using cutting tools Such an intervention is carried out routinely in the case of retinal detachments or the peeling of epiretinal membranes, but is usually considered a disproportionate therapy for removing the localized vitreous humor opacification. Moreover, vitrectomy is invasive, requires a stay at a clinic and harbors the risks linked to surgical interventions, in particular the frequent inducement of a cataract, seldom a retinal detachment and very seldom, but possibly, endophthalmitis.

So-called laser vitreolysis now offers a low-risk treatment alternative. Laser vitreolysis is a sparing, low-risk and pain-free laser treatment, by application of which the vitreous humor opacification can be vaporized or atomized without opening up the eye.

In the case of laser vitreolysis, short laser light pulses are directed at the vitreous humor opacification in order to obtain optical breakdown or photodisruption there on account of the high laser intensity in the focal region.

The floaters and the vitreous humor surrounding these absorb the laser energy and a cutting or expanding laser plasma is formed, as a result of which the floaters are vaporized and/or comminuted and can as a result dissolve. The treatment causes little pain and is without risk of infection. Laser vitreolysis provides a safe method for the sparing treatment of bothersome vitreous humor opacification should it be possible to ensure that important and sensitive eye structures, for example the capsular bag, the crystalline lens or retinal regions, especially the macula, are not damaged by the laser.

However, the success of the treatment depends on the type of floater. The treatment is particularly successful in the case of so-called Weiss rings. Tissue strands can be severed and the tissue concentrations responsible for the disturbing shadows can be eradicated.

Floaters have already been treated with YAG lasers (in particular as Nd:YAG at 1064 nm) for more than three decades (Brasse, K., Schmitz-Valckenberg, S., Junemann, A. et al. Ophthalmologe (2019) 116: 73. https://doi.org/10.1007/s00347-018-0782-1). However, only the most anterior region of the vitreous humor can be treated with precision and reliable targeting, even when the current high-end devices are used. These lasers are not precise enough in the deeper vitreous humor region. However, most vitreous humor opacification is found there as this often is the result of posterior vitreous humor detachment. YAG lasers are often used in ophthalmology for iridotomy in glaucoma diseases and for post-cataract treatment, that is to say to remove opacification or even a post-cataract membrane on artificial lens implants as a result of cell overgrowth. Frequency-doubled YAG lasers with laser radiation in the green range (532 nm) are also used for retinal coagulation, for example in the event of bleeding or retinal detachment. YAG lasers are less frequently also used for phacoemulsification in cataract surgery, that is to say for the liquefaction of the clouded and hardened natural lens. In this case, however, this tends to be an Er:YAG laser with a wavelength of 2940 nm and higher water or tissue absorption, which then often has to be laboriously introduced into the eye by use of an endoscopic laser introduction with a mirror guide.

According to the known prior art, there are already the numerous solutions for carrying out laser surgery on the tissue of the eye, especially in the vitreous humor.

Thus, DE 10 2011 103 181 A1 describes an apparatus and a method for femtosecond laser surgery on tissue, especially in the vitreous humor of the eye. The apparatus consists of an ultrashort pulse laser with pulse lengths ranging from approximately 10 fs-1 ps, in particular approximately 300 fs, pulse energies ranging from approximately 5 nJ-5 μJ, in particular approximately 1-2 μJ, and pulse repetition rates of approximately 10 kHz-10 MHz, in particular 500 kHz. The laser system is coupled to a scanner system which allows the spatial variation in the focus in three dimensions. In addition to this therapeutic laser scanner optics system, the apparatus furthermore consists of a navigation system coupled therewith.

US 2006/195076 A1 describes a system and method for producing incisions in ocular tissue at various depths. The system and the method focus light, possibly in a pattern, on different foci situated at a different depths within the ocular tissue. A plurality of foci can be created simultaneously by way of a segmented lens. Optimal incisions can be obtained by virtue of the light being focused at different depths, either successively or simultaneously, and an extended plasma column and a beam with a lengthened waist being generated. The techniques described in this case can also be used, inter alia, to perform novel ophthalmological methods or to improve existing methods, including dissection of tissue in the posterior pole, for example floaters, membranes and the retina.

US 2014/257257 A1 also describes a system and its method for treating target tissue in the vitreous humor of an eye, comprising a laser unit for producing a laser beam and a detector for producing an image of the target tissue. The system also contains a computer which defines a focal spot path for emulsifying the target tissue. A comparator connected to the computer then controls the laser unit in order to move the focus of the laser beam. This focus movement is carried out to treat the target tissue while deviations of the focus from a defined focus path are minimized.

US 2015/342782 A1 likewise relates to a system and a method for using a computer-controlled laser system, for carrying out a partial vitrectomy of the vitreous humor in an eye. Operationally, an optical channel through the vitreous humor is defined first. Vitreous-like and suspended depositions (floaters) in the optical channel are then ablated and removed from the optical channel (e.g., aspirated) in some cases. In some cases, a clear liquid can be introduced into the optical channel in order to replace the ablated material and thereby establish an unimpeded transparency in the optical channel. In general, the present invention relates to systems and methods for ophthalmological laser operations. In particular, the present invention relates to systems and methods for using pulsed laser beams for removing what are known as floaters.

US 2018/028354 A1 likewise describes a method and a system for an ophthalmological intervention in an eye. Unwanted features are identified on the basis of an image of at least a portion of the eye.

Unwanted features in the vitreous humor cavity are considered to be instances of vitreous opacification that impair sight, for example floaters. After the floaters have been identified and localized by an image processing system, they are automatically “shot” with laser pulses following confirmation by a physician. The laser energy evaporates at least some of the vitreous-like opacity. This procedure is repeated until the opacification of the vitreous humor has been removed. The entire procedure is repeated for each instance of opacification in the vitreous humor until the liquid of the vitreous humor is considered to be sufficiently clear.

A method described by ELLEX (product brochure by Ellex Medical Pty Ltd.; “Tango Reflex—Laser Floater Treatment”; PB0025B, 2018; (http://www.ellex.com)) provides for the use of a pulsed nanosecond laser (YAG) in order to decompose vitreous humor opacification or completely remove the latter by a transition into a gas. A pilot laser beam is used to sight the target area (floater), which is subsequently “shot” using one or more therapeutic laser pulses. In this case, both the pilot laser beam and the therapeutic laser pulse are manually triggered by the user. Such a manual laser treatment typically consists of two individual treatments, each having a duration of 20-60 minutes.

Applications DE 10 2019 007 147.6 and DE 10 2019 007 148.4, which have not yet been published, describe systems for laser vitreolysis of floaters, which enable the safe and precise atomization of vitreous opacification (floaters) on the basis of a combination of a treatment laser with an OCT or an OCDR system. Here, an OCDR (optical coherence domain reflectometry) system is understood to mean a system for interferometric acquisition of one-dimensional scattering profiles, while OCT (optical coherence tomography) is understood to mean 2-dimensional or 3-dimensional imaging. In both cases variants with recording sequences (i.e., film) should also be included. In the process, minimum distances to sensitive eye structures are ensured and the activation of the laser is preferably only permitted if the focus of the treatment laser and the floater to be treated are positioned with sufficient accuracy in relation to one another.

A disadvantage of both approaches is that the physician has to constantly combine the available, familiar 2-dimensional frontal view of the eye with the 3-dimensional recordings of the OCT or OCDR system using their spatial imagination, which is extremely difficult in the context of the time-critical or quick interactions with the patient.

### SUMMARY OF THE INVENTION

The use of laser energy within the scope of laser vitreolysis is non-invasive and avoids the disadvantages of surgical interventions, but is also linked to disadvantages and risks.

For example, targeting the laser may be difficult. Since the physician observes the vitreous humor along the beam path, it may be difficult to determine the depth of the position of the retina, the depth of the opacification of the vitreous humor or other relevant features. As a consequence, there is the risk of the opacification of the vitreous humor being missed and/or the eye being injured.

For example, the treatment of largely transparent floaters, which change in position and are difficult to recognize but nevertheless, as phase objects, are able to generate bothersome shadows on the retina, was found to be difficult.

The application of laser energy may also lead to an additional movement of the opacification of the vitreous humor, making the treatment even more difficult. Thus, the physician may need to realign the laser after each application of laser energy. This may require much time. Therefore, a treatment with laser energy is complicated and causes stress, both for the patient and for the physician.

A further possible problem relates to incomplete vitreous humor detachment, which may lead to local vitreous traction right up to retinal detachment. Laser treatment in the vitreous humor can lead to changes in the balance of forces in the vitreous body due to shockwaves propagating as a consequence of said treatment, and thereby for example cause tension on the retina.

Lastly, the treatment of those floaters situated in the vicinity of sensitive structures of the eye was also found to be particularly difficult. In this case, the laser radiation can lead to damage in the retina, the crystalline lens or the macula.

Example embodiments of the present invention facilitate a solution for laser treatment of eye opacification that eliminates the disadvantages of the known technical solutions and offers a possibility of combining 2-dimensional views of the eye with the 3-dimensional recordings of the measuring system in a simple way, thus easing the handling thereof for the operator. Moreover, example embodiments are easy to implement and economically cost-effective, and enable a simpler, faster, and, above all, safer treatment of bothersome vitreous humor opacification by way of laser vitreolysis.

Using the arrangement for laser treatment of eye opacification according to example embodiments of the invention, including a measuring system for obtaining depth information relating to eye structures, a laser system with optical elements for coupling the measuring and laser system, an eye tracker unit, a display unit, and a control and operating unit, facilitates treatment by virtue of the fact that the measuring system is designed to make available depth information relating to eye structures in the form of depth profiles, the laser system is designed to comminute eye opacification, the eye tracker unit is designed to detect axial eye movements, the display unit is designed to display at least one 2-D image representation of the eye as a live image, the control and operating unit is designed to determine the depth of eye structures relative to the depth of the laser focus from the depth profiles and to determine an exclusion zone for laser treatment, in particular for the retina and the capsular bag, and the control and operating unit is further designed to generate at least one marking for at least each of the exclusion zones of the retina and capsular bag and the laser focus, the characteristics of which markings correspond to the respective depth in the eye, in order to display these markings on the display unit and overlay these on the live image.

In this case, the deflection unit can be realized using known electromechanical deflection mirrors (e.g., galvo or MEMS scanners) or by a simple manually operated deflection and/or displacement of the laser beam, for example using a hand-held contact glass and a displaceable and pivotable laser slit lamp.

According to example embodiments of the invention, the control and operating unit is configured to localize floaters and structures of the eye and to determine an exclusion zone for laser treatment, for example for the retina and the capsular bag. Furthermore, the control and operating unit is configured to generate a marking for at least the exclusion zones of the retina and capsular bag and the laser spot, the size of which markings correspond to the respective depth information in the eye, in order to display these markings on the display unit and overlay these on the live image and/or the scan.

A first example embodiment relates to the measuring system, which is an OCDR or OCT system, for acquiring depth information of the eye.

A second group of example embodiments relates to the eye tracker unit, with anterior regions of the eye, preferably the iris, capsular bag, lens or front or back surface of the cornea, a contact glass or a reference marking placed in the vitreous humor by laser treatment serving as a reference for said eye tracker unit.

A third group of example embodiments relates to the display unit, which is designed to display further image representations, for example the scan with the depth information of the eye and/or an overview of current settings and/or operating elements. For example, the display unit is a touchscreen in this case.

Further example configurations relate to the control and operating unit, which is also designed to localize retinal landmarks, for example the fovea, macula, optic nerve head or else blood vessels, and to generate a marking in order to additionally display these on the display unit for laser treatment.

The markings generated by the control and operating unit in this case differ in terms of color and/or structure. For example, the marking generated for the laser spot changes its color and/or structure when the laser spot approaches or enters one of the exclusion zones or retinal landmarks.

The control and operating unit is further configured to switch off the laser system should the laser focus approach or enter one of the exclusion zones or retinal landmarks. Different tolerances for switching off the laser system on approach or entry can be assigned to the two exclusion zones and the retinal landmarks.

According to a further example embodiment, the arrangement for laser vitreolysis of vitreous humor opacification is integrated into a slit lamp.

Example embodiments of the present invention relate to an arrangement for laser treatment of eye opacification. A partially automated therapy device (system) is proposed, in which 2-dimensional views of the eye are combined with the depth information (depth profiles) in order to make it easier for an operator to use when localizing the floaters over the course of the treatment.

### BRIEF DESCRIPTION OF THE DRAWINGS

The invention is described in more detail below on the basis of example embodiments. In the figures:

FIG. 1: depicts a fundus image with overlaid markings,

FIG. 2: depicts a fundus image with overlaid markings for different focal positions of the laser spot, and

FIG. 3: depicts a scan with markings for the exclusion areas and the laser spot.

### DETAILED DESCRIPTION

The example arrangement for laser treatment of eye opacification arrangement for laser vitreolysis of vitreous humor opacification includes a measuring system for obtaining depth information of the eye, a laser system with deflection unit, optical elements for coupling the measuring and laser system, an eye tracker unit, a display unit, and a control and operating unit.

For example, the measuring system is designed to provide depth information of the eye in the form of scans. Axial eye movements are detected and compensated for by the eye tracker unit. The display unit is designed to display at least one 2-D image representation of the eye as a live image. Refresh rates of 5 Hz, for example 10 Hz, and in another example 20 Hz, and a latency <200 ms, for example <100 ms, and in another example <35 ms, are used for the live image.

According to an example embodiment of the invention, the control and operating unit is configured to determine the depth of eye opacification relative to the depth of the laser focus and to generate markers for the relative position of eye opacification in relation to the laser focus and to display these markings on the display unit.

Furthermore, the control and operating unit is configured to localize structures of the eye in the scans made available by the measuring system and to determine an exclusion zone for laser treatment, for example for the retina and the capsular bag.

For example, the control and operating unit is further configured to generate a marking for at least the exclusion zones of the retina and capsular bag and the laser spot, the size of which markings corresponds to the respective depth information in the eye, to display these markings on the display unit and overlay these on the live image.

By determining the position of the laser focus in relation to the eye structures to be protected exclusion zones generated for these eye structures, the physician is given the option of treating floaters manually. The generated exclusion zones prevent the laser focus from entering and damaging eye structures.

In accordance with an example embodiment, the control and operating unit further is configured to determine the depth of eye opacification relative to the depth of the laser focus and to generate markers for the relative position of eye opacification in relation to the laser focus and to display these markings on the display unit.

By additionally determining the position of eye opacification (floaters) in relation to the eye structures to be protected or in relation to the exclusion zones generated for these eye structures, there is the option of automatically treating floaters.

A manual treatment that is likewise still possible is simplified by virtue of the physician being able to at least approximately estimate the position of the laser focus in relation to the floaters.

In the arrangement presented herein, an OCDR or OCT system is used as the measuring system for obtaining depth information of the eye, said measuring system for example being combined with a YAG laser system with a deflection unit.

An A-scan and, with the aid of a further image recording unit, additional recordings of the fundus are recorded when an OCDR system is used, with the position of the OCDR measuring beam in relation to the fundus being known through calibration. The recordings of the fundus are for example recorded using IR or NIR illumination between 780 nm and 1060 nm.

In contrast thereto, a 3-D volume scan is recorded when the OCT system is used, with the eye movements being detected and compensated for by the eye tracker unit during the recording of the 3-D volume scan. Eye movement compensation is required because a 3-D volume scan acquisition may take up to 2 s and eye movements would cause the scans to be deformed.

While the eye structures can be detected relatively easily in the OCDR or OCT signals, the situation is different with floaters.

The identification is for example carried out in such a way that the scattering signal level of the vitreous humor is determined first. Structures which
have signal values that are 2 dB, for example 5 dB higher than the average vitreous body signal,
have a minimum axial size, for example >15 μm in tissue or an equivalent optical path with an assumed refractive index of 1.36 and in the case of which signals still occur posteriorly at the level of the vitreous body or
whose signal characteristics correspond to known floaters stored in a database, using size or position ratios, are identified as floaters. The eye tracker unit is designed to detect and compensate for the axial eye movements.

For example, the anterior regions of the eye serve as a reference, for example the capsular bag, lens or front or back surface of the cornea.

Since the retina can be more shadowed by floaters, it is not usually used as a reference. Therefore, more anterior eye regions are for example preferred for tracking the axial eye movement in relation to the floaters.

However, it is also possible for the contact glass or a reference marking placed in the vitreous humor by laser treatment to be used as a reference for the eye tracker unit. Should the contact glass be used as a reference, referencing in the form of a functional coating is conceivable to allow stable tracking.

According to the invention, the display unit is designed to display further image representations, for example the scan with the depth information of the eye and/or an overview of current settings and/or operating elements, in addition to a 2-D image representation of the eye as a live image. In this context, the display unit can be a touch screen.

According to example embodiments of the invention, binoculars, 3-D monitors, HMDs or the like are also provided as a display unit. Furthermore, the design of the control and operating unit is important to the invention. For example, it generates markings for the exclusion zones of the retina and capsular bag and the laser spot, in order to display these on the display unit and overlay them on the live image and/or scan. Even the floater itself can be provided with a marking in the process.

The control and operating unit is further developed to localize retinal landmarks, such as the fovea, macula, optic nerve head or else blood vessels, and to generate a respective marking for each of these in order to optionally also display these on the display unit and overlay these on the live image/and or scan.

To avoid injury, the laser treatment can be interrupted if the laser beam is aimed at one of the retinal landmarks.

Moreover, according to the invention, provision is made for local changes in the height of the retina to be monitored, for the purposes of which critical local points can likewise be marked, as described, in order to avoid stresses that could cause retinal detachments during a further laser treatment.

According to an example embodiment the invention, the markings generated by the control and operating unit differ in terms of color and/or structure.

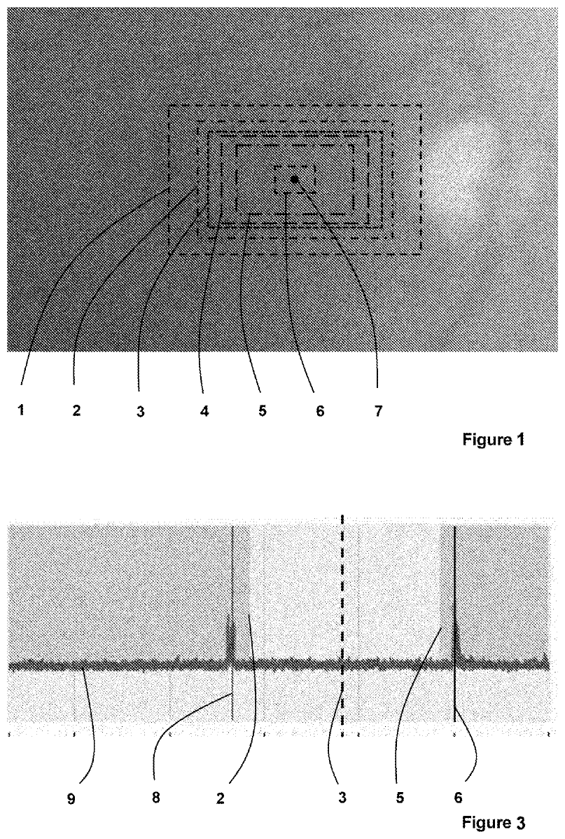

In this respect, FIG. 1 shows the representation of a fundus image with overlaid markings, with both the fundus image and the markings being colored in reality. From outside to inside, the markings denote: capsular bag 1, anterior exclusion zone boundary 2, laser focus 3, floaters 4, posterior exclusion zone boundary 5, retina 6, and laser direction 7.

Furthermore, the marking generated by the control and operating unit for the laser spot changes its color and/or structure when the laser spot approaches one of the exclusion zones or retinal landmarks.

It is also possible to warn the operator acoustically and/or optically or switch off the laser system should the laser focus approach or enter one of the exclusion zones or retinal landmarks.

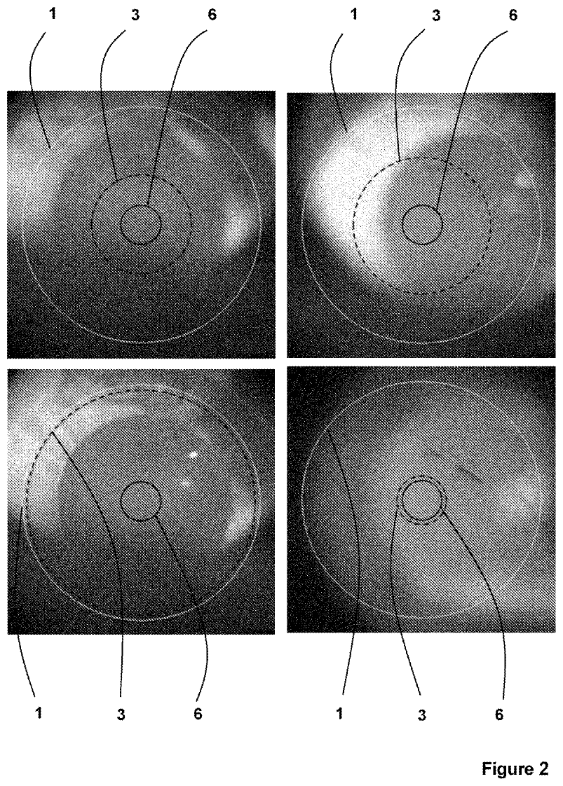

In this respect, FIG. 2 shows the representation of a fundus image with overlaid markings for different focal positions of the laser spot, with both the fundus image and the markings in this case also being colored in reality. In the fundus images depicted here, only the markings for the retina 6, the laser focus 3 and the capsular bag 1 are present for better clarity. While a laser treatment is possible in the top two figures because the laser focus 3 is located in the “safe” area between the retina 6 and capsular bag 1, a laser treatment is not possible in the two lower images, by contrast, because the laser focus 3 comes too close to the retina 6 or the capsular bag 1 and is located in the respective exclusion zone (not shown) in each case.

FIG. 3 shows the representation of a scan with markings for the exclusion areas and the laser spot. From this representation of the depth scan 9, the user can very quickly and reliably identify the depth at which the laser focus 3 is located in relation to the retina 6 and the lens 8 (or the capsular bag 1). Additionally, the posterior exclusion zone 5 and the anterior exclusion zone 2 are also depicted here.

Furthermore, the control and operating unit is designed to assign different tolerances for the approach to both exclusion zones and the retinal landmarks.

In an example embodiment, the arrangement for laser vitreolysis of vitreous humor opacification is integrated into a slit lamp. However, the concept proposed here for laser vitreolysis is likewise applicable in a similar way in the case of surgical microscopes.

The example arrangement is also suitable for displaying a type of treatment history, for example by marking and storing shot depths and shot positions. It is also possible to display the incremental change in position of floaters (as a trajectory) and also retinal portions when shooting incrementally with the laser (marking of local elevations).

Example embodiments of the invention make available an arrangement for laser treatment of eye opacification that eliminates the disadvantages of the known technical solutions and offers a possibility of combining 2-dimensional views of the eye with the 3-dimensional recordings of the measuring system in a simple way, thus easing the handling thereof for the operator. Moreover, the solution is easy to implement and economically cost-effective, and enables a simpler, faster, and, above all, safer treatment of bothersome vitreous humor opacification by way of laser vitreolysis.

According to example embodiments of the invention, the depth information with regard to the position of sensitive eye structures, the laser focus, and the possibly also moving floater to be processed obtained from OCDR or OCT systems are processed in such a way that they can be combined with the 2-D view familiar to the physician in an intuitively processable manner.

1.-21. (canceled)

22. An arrangement for laser treatment of eye opacification, comprising:
a measuring system that obtains depth information relating to eye structures;
a laser system with optical elements that couple the measuring system and the laser system;
an eye tracker unit;
a display unit; and
a control and operating unit;
wherein the measuring system is configured to make available depth information relating to eye structures in the form of depth profiles;
wherein the laser system is configured to comminute eye opacifications;
wherein the eye tracker unit is configured to detect axial eye movements;
wherein the display unit is configured to display at least one 2-D image representation of the eye as a live image;
wherein the control and operating unit is configured to determine a depth of eye structures relative to a depth of a laser focus from the depth profiles and to determine at least one exclusion zone for laser treatment, the exclusion zone including at least one of a retina and a capsular bag, and
wherein the control and operating unit is further configured to generate at least one marking that marks each of the at least one exclusion zones and marks the laser focus, characteristics of the at least one marking corresponding to a respective depth in the eye, and configured to display the markings on the display unit and to overlay the markings on the live image.

23. The arrangement as claimed in claim 22, wherein the control and operating unit is configured to determine the depth of the eye opacification relative to the depth of the laser focus and to generate markers for the relative position of eye opacification in relation to the laser focus and to display these markings on the display unit.

24. The arrangement as claimed in claim 23, wherein the control and operating unit is configured to vary characteristics of the marking to be generated with regard to at least one of color, shape, numerical value of the depth and size.

25. The arrangement as claimed in claim 22, wherein the control and operating unit is configured to generate the markings with depth-dependent size for the retina, the capsular bag, a lens, the laser focus or the eye opacification in such a way that the markings are arranged, centered around the lateral laser focus position.

26. The arrangement as claimed in claim 22, wherein the eye tracker unit is configured to compensate for axial eye movements.

27. The arrangement as claimed in claim 22, wherein the one 2-D image representation is an en face image representation of the posterior segment of the eye which is displayed as a live image with a refresh rate selected from a group consisting of 5 Hz, 10 Hz, and 20 Hz, and a latency selected from a group consisting of <200 ms, <100 ms and <35 ms.

28. The arrangement as claimed in claim 22, wherein the measuring system for obtaining depth information of the eye comprises an OCDR system.

29. The arrangement as claimed in claim 22, wherein anterior regions of the eye, selected from a group consisting of the capsular bag, a lens, a front surface of the cornea or back surface of the cornea, serve as a reference for the eye tracker unit.

30. The arrangement as claimed in claim 22, wherein a contact glass or a reference marking placed in the vitreous humor by laser treatment serves as a reference for the eye tracker unit.

31. The arrangement as claimed in claim 22, further comprising an OCDR system configured to record an A-scan and further comprising an image recording unit for making recordings of the fundus, with a position of the OCDR measuring beam in relation to the fundus being known through calibration.

32. The arrangement as claimed in claim 22, further comprising an OCT system configured to record a 3-D volume scan and wherein the eye tracker unit is configured to compensate for eye movements detected during the recording of the 3-D volume scan.

33. The arrangement as claimed in claim 22, wherein the display unit is configured to display further image representations, including the scan with the depth information of the eye, an overview of current settings, operating elements or a combination of the foregoing.

34. The arrangement as claimed in claim 22, wherein the display unit comprises a touch screen.

35. The arrangement as claimed in claim 22, wherein the control and operating unit is configured to display on the display unit the markings generated for the exclusion zones of the retina and capsular bag and for the laser focus and to overlay these on the scan.

36. The arrangement as claimed in claim 22, wherein the control and operating unit is configured to localize retinal landmarks, including at least one of a fovea, a macula, an optic nerve head and blood vessels, and to generate a marking for the retinal landmarks.

37. The arrangement as claimed in claim 23, wherein the markings generated by the control and operating unit differ in terms of color, structure or both.

38. The arrangement as claimed in claim 36, wherein the marking generated by the control and operating unit for a laser spot changes color, structure or both when the laser spot approaches one of the exclusion zones or the retinal landmarks.

39. The arrangement as claimed in claim 22, wherein the control and operating unit is configured to warn the operator acoustically, optically or both when the laser focus approaches one of the exclusion zones or retinal landmarks.

40. The arrangement as claimed in claim 22, wherein the control and operating unit is configured to switch off the laser system should the laser focus approach or enter one of the exclusion zones or retinal landmarks.

41. The arrangement as claimed in claim 22, wherein the control and operating unit is configured to assign different tolerances for switching off the laser system when the laser focus is approaching the two exclusion zones and the retinal landmarks.

42. The arrangement as claimed in claim 22, wherein the arrangement for laser treatment of eye opacification is integrated into a slit lamp.
